(19) United States
(12) Patent Application Publication
(10) Pub. No.: US 2012/0133887 A1
(43) Pub. Date: May 31, 2012

(54) TEAR FILM AND TEAR MENISCUS DYNAMICS WITH TIME-LAPSE OPTICAL COHERENCE TOMOGRAPHY

(75) Inventor: David HUANG, Portland, OR (US)

(73) Assignee: Optovue, Inc., Fremont, CA (US)

(21) Appl. No.: 13/308,152

(22) Filed: Nov. 30, 2011

Related U.S. Application Data
(60) Provisional application No. 61/418,324, filed on Nov. 30, 2010.

(51) Int. Cl. A61B 3/14; G01B 9/02

(52) U.S. Cl. 351/206; 356/479; 351/246

(57) ABSTRACT

In accordance with some embodiments of the present inventions, an imaging device includes an OCT imager, a trigger, a computer coupled to the OCT imager and the trigger, the computer executing instructions for: generating a first signal at the trigger to initiate closing of an object at a first time, generating a second signal at the trigger to initiate opening of an object at a second time following the first time, acquiring a plurality of OCT data scans with the OCT imager at different time intervals following the second time, identifying an area of interest in the plurality of OCT data scans, identifying layers in the area of interest, calculating thickness measurements of the layers from the OCT data scans, and displaying the thickness measurements.

### RELATED APPLICATIONS

This application claims priority to U.S. Provisional Application 61/418,324, filed on Nov. 30, 2010, which is herein incorporated by reference in its entirety.

### BACKGROUND

1. Field of Invention

The embodiments described herein relate to methods and apparatus in the field of medical imaging, and in particular to methods and apparatus for acquiring and processing optical coherence tomography (OCT) of tear film and tear meniscus to measure post-blink dynamics and assess dry eye severity.

2. Background State of the Arts

Optical coherence tomography (“OCT”) is a high-resolution imaging technology that is used for in vivo cross-sectional and 3D imaging of microstructure in biological tissues for more than two decades (Huang D, et al, [Science. 254, 1178-1181 (1991)]). Imaging systems using OCT technologies have been used to image central tear film thickness and upper and lower tear menisci in subject eyes. (See for example, Wang et al., [Invest Ophthalmol Vis Sci 47, 4349-4355 (2006)], Palakuru J R et al., [Invest Ophthalmol Vis Sci 48, 3032-3037 (2007)], or Zhou et al., [Ophthalmic Surg Lasers Imaging 40, 442-447 (2009)]). However, because tear film is a very thin liquid layer, attempts were made to estimate thickness of the tear film using an indirect technique (Wang et al., [Invest Ophthalmol Vis Sci 47, 4349-4355 (2006)]). In this technique, the tear film was measured as the difference between the combined tear film-cornea thickness minus the corneal thickness. The corneal thickness (epithelium to endothelium) was separately measured after instillation of artificial tear in order to increase the tear film thickness and to allow the thickness to be separately measured. This method is highly susceptible to errors in corneal thickness measurement because the cornea is about 200 times thicker than the tear film. Tear dynamics after a blink have also been measured by the operator measuring the tear film and tear meniscus at various times after different blinks (Palakuru J R et al., [Invest Ophthalmol Vis Sci 48, 3032-3037 (2007)]). But this approach does not measure the tear dynamics of a single blink motion, the dynamics of tear film formation and breakup are therefore confounded by blink-to-blink differences. Other techniques were developed to evaluate tear meniscus measured at a fixed time after a blink using high-speed Fourier-domain OCT (Zhou et al., [Ophthalmic Surg Lasers Imaging 40, 442-447 (2009)]). However, these techniques do not provide accurate measurement of the tear film or evaluation of the tear dynamics.

Thus there is a need for better OCT acquisition and processing algorithms to capture the blink dynamics of both the tear film and tear meniscus of the eye.

### SUMMARY

This Summary is provided to briefly indicate the nature and substance of the invention. It is submitted with the understanding that it will not be used to interpret or limit the scope or meaning of the claims.

In accordance with some embodiments of the present inventions, an imaging device includes an OCT imager, a trigger, a computer coupled to the OCT imager and the trigger, the computer executing instructions for: acquiring a plurality of OCT data scans with the OCT imager at different time intervals, generating a first signal at the trigger to initiate closing of an object at a first time, generating a second signal at the trigger to initiate opening of an object at a second time following the first time, identifying an area of interest in the plurality of OCT data scans, identifying layers in the area of interest, calculating thickness measurements of the layers from the OCT data scans, and displaying the thickness measurements.

A method according to some embodiments of the present invention includes acquiring a plurality of OCT data scans at different time intervals, generating a first signal to initiate closing of an object at a first time, generating a second signal to initiate opening of an object at a second time following the first time, identifying an area of interest in the plurality of OCT data scans, identifying layers in the area of interest, calculating thickness measurements of the layers from the OCT data scans, and displaying the thickness measurements.

These and other embodiments are further described below with respect to the following figures.

### BRIEF DESCRIPTION OF THE DRAWINGS

FIGS. 1a and 1b show an exemplary circular optical coherence tomography (OCT) scan of the central corneal tear film and its cross sectional image.

FIGS. 2a and 2b show a magnified OCT image and a selected axial scan (A-scan) from the data in FIGS. 1a and 1b.

FIGS. 3a, 3b, and 3c show a representation of a vertical OCT scan and its corresponding cross-sectional OCT images of the lower tear meniscus.

FIG. 4 shows an exemplary illustration of a timing diagram for time-lapse OCT of the tear film or tear meniscus.

FIG. 5 is a schematic illustration of an optical coherence tomography (OCT) scanner.

FIG. 6 illustrates an embodiment of an acquisition procedure according to some embodiments of the present invention.

### DETAILED DESCRIPTION

OCT technologies have been commonly used to obtain high resolution images in the medical field for over two (2) decades, especially in the field of ophthalmology. High quality and real-time cross sectional images and 3D data sets of the eye using OCT technologies are capable of producing high resolution structural images of the eye suitable for clinical interpretation and diagnosis of different eye diseases and conditions. The advancement from time-domain OCT technology to Fourier-domain technology has further enhanced these advantageous characteristics of this imaging modality. (See for example, Wojtkowski M. et al, [Opt. Lett. 27, 1415-1417 (2002)], Leitgeb R. et al, [Opt. Express 11, 889-894 (2003)], and De Boer J F., [Opt. Lett. 28, 2067-2069 (2003)]).

Exemplary embodiments of the present invention generally include methods and systems to image and analyze the dynamics of tear film and tear meniscus of a subject eye. In some embodiments of the present invention, a Fourier-domain OCT system is used to perform a circular scan of the central cornea for the measurement of the central tear film. In some embodiments, a vertical line scan of the lower tear meniscus is imaged. Each scan is performed consecutively several times within a small fraction of a second to improve image quality using frame-averaging. Tear film or tear meniscus imaging is performed as a time lapse series of approximately 13 seconds. The time lapse series imaging starts with a signal for the subject to close the eye. This is followed by a command for the subject to open the eye 1 to 2 seconds after the initial signal to close. This blink motion allows the dynamics of the tear film and tear meniscus to be captured when the subject's eye closes and opens. According to further aspects of the present invention, the OCT series can be analyzed to measure the tear film+epithelial thickness and tear meniscus cross-sectional area as a function of time after blink. The tear film+epithelial thickness time profile can be analyzed to obtain total change and half-time measurements. Similarly, the tear meniscus cross-sectional area time profile can be analyzed to obtain the initial meniscus area after blink, total change and half-time.

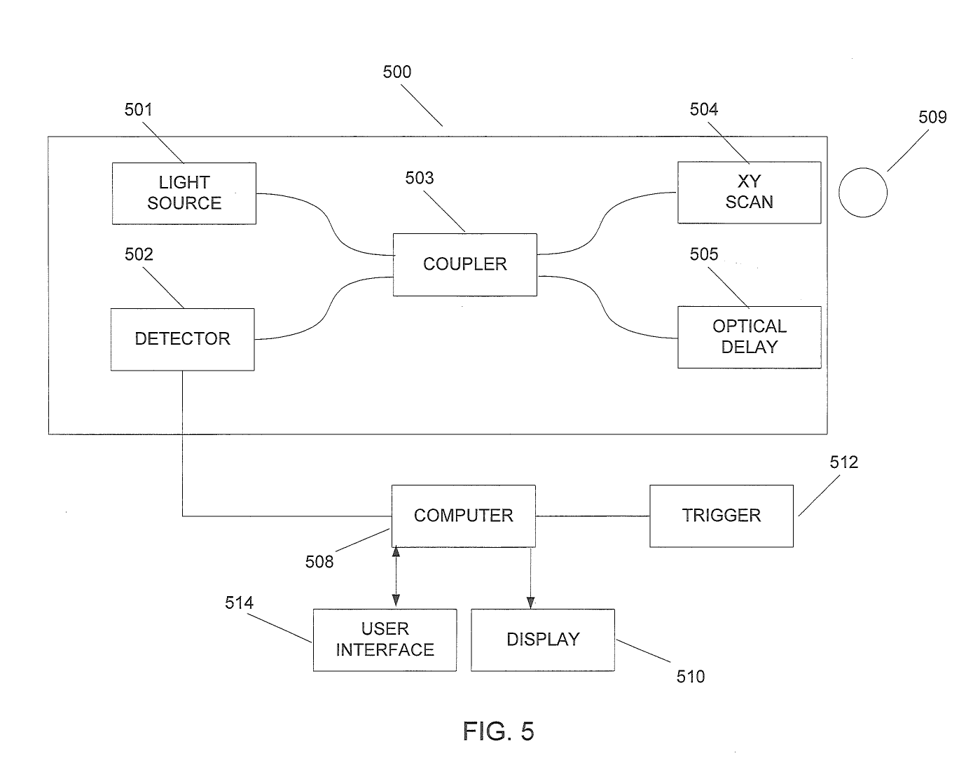

FIG. 5 illustrates an example of an OCT imager 500 that can be utilized in imaging and analyzing tear film and tear meniscus dynamics according to some embodiments of the present invention. OCT imager 500 includes light source 501 supplying light to coupler 503, which directs the light through the sampling arm to XY scan 504 and through the reference arm to optical delay 505. XY scan 504 scans the light across eye 509 and collects the reflected light from eye 509. Light reflected from eye 509 is captured in XY scan 504 and combined with light reflected from optical delay 505 in coupler 503 to generate an interference signal. The interference signal is coupled into detector 502. OCT imager 500 can be a time domain OCT imager, in which case depth (or A-scans) are obtained by scanning optical delay 505, or a Fourier-domain imager, in which case detector 502 is a spectrometer that captures the interference signal as a function of wavelength. In either case, the OCT A-scans are captured by computer 508, which is coupled to detector 502 and may be coupled to control XY scan 504, optical delay 505, and light source 501. Collections of A-scans taken along an XY pattern are utilized in computer 508 to generate 3-D OCT data sets. Computer 508 can also process the 3-D OCT data sets into 2-D images according to some embodiments of the present invention.

Computer 508 can be any device capable of processing data and may include any number of processors or microcontrollers with associated data storage such as memory or fixed storage media and supporting circuitry. Computer 508 can be coupled to a display 510 and a user interface 514. User interface 514 and display 510 allow for an operator to communicate and control computer 508, and by extension OCT imager 500. In some embodiments, computer 508 may communicate with another processor coupled with OCT imager 500 that controls OCT imager 500 under the direction of computer 508. Computer 508 can also be coupled to a trigger signaling device 512 that interfaces with sample 509. For example, sample 509 may be a patient's eye and trigger signaling device 512 may alert the patient to either close or open the eye.

According to some embodiments of the present invention, OCT 500 may be a high-speed OCT system utilizing Fourier-domain technology. Current commercial ophthalmic Fourier-domain OCT systems operate at speeds of 17,000 to 40,000 axial scans (A-scans) per second. The next generation of Fourier-domain OCT systems, currently available in research laboratories, are likely to operate at an even higher rate of 70,000 to 100,000 A-scans per second. In the following descriptions, an OCT system with a scan rate of 70,000 A-scans per second is used to illustrate embodiments of OCT 500. However, according to some embodiments, the present invention can be applied to Fourier-domain OCT system with different scan rate; time-domain OCTs with any scan speed can be used with embodiments of the present invention.

Tear Film Imaging

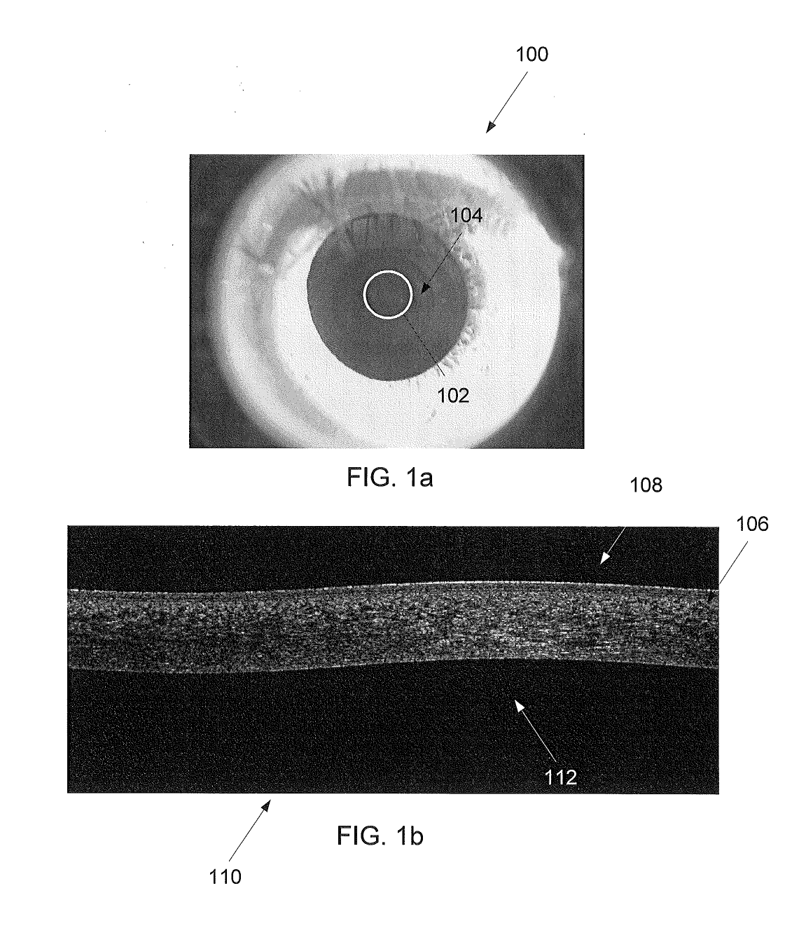

FIG. la shows an exemplary circular OCT scan of a central corneal tear film 100. The resultant cross sectional image 110 illustrated in FIG. 1b is the cross sectional image of central corneal tear film 100. Image 100 is from a video image of an OCT system showing the anterior segment of a subject eye. Video image 100 can be used to show a 2-mm diameter OCT scan path 102 centered on the pupil 104 (dark circular region in the video image 100). This circular transverse scan path 102 yields a cylindrical cross-section that can be then unfolded and displayed. For example, image 110 is a gray-scale representation of such cross-sectional image. The vertical dimension of image 110 is the depth or thickness of an A-scan and the horizontal dimension is the circumferential dimension of the circular scan 102 shown in FIG. 1a. In the vertical direction, the anterior side 106 of the cornea of the subject eye faces upward (with the air-eye interface 108 at the top and the interior of the eye 112 at the bottom). In image 110, a gray scale representation was used to display the OCT signal strength as brightness in a logarithmic scale; the higher the signal strength, the more light is reflected from a tissue layer and the brighter the pixel in the image 110. In some embodiments, the OCT circular scan 102 is composed of a large number of scans, for example approximately 1024 A-scans. At a scan rate of 70,000 A-scan per second, one circular scan can be completed in less than 0.015 seconds; motion error is likely to be insignificant within such a short acquisition period.

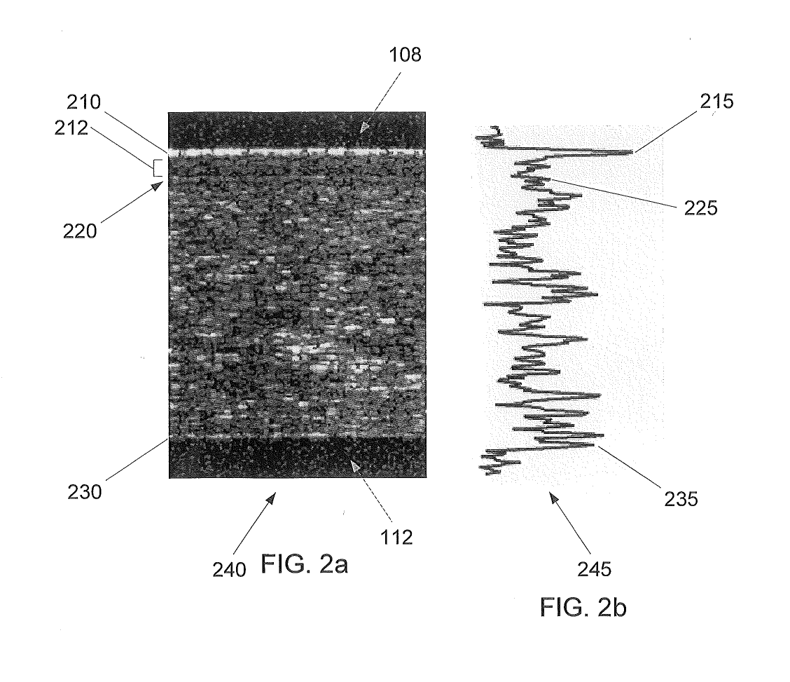

FIG. 2a shows a magnified OCT image 240 and FIG. 2b shows a selected A-scan 245 extracted from the image 110 in FIG. 1b. Image 240 is a magnified section of the OCT cross-section image 110 focused at the corneal layers, with the air 108 at the top and the anterior chamber of the eye 112 at the bottom. An air-tear interface 210 reflects significant amount of light and produces a very bright horizontal band as shown in image 240. The Bowman's layer, located between the superficial epithelium and the stroma in the cornea, produces two thin bright lines 220. The epithelial layer 212 is shown as the low reflectance layer between the air-tear interface 210 and the Bowman's layer 220. There are low amplitude speckle noise peaks within the epithelium 212. The speckle appears random and can be distinguished from the brighter anterior Bowman's layer boundary reflection 220 which forms a continuous line. The endothelium layer 230 is shown as the last bright line (deepest in the vertical dimension) before entering the anterior chamber of the eye 112. The OCT image 240 is a magnified image of the OCT cross-section image 110, which is composed of many A-scans over a range of transverse locations. One of such A-scan is selected and displayed as a waveform image 245 in FIG. 2b. In the waveform image 245, the vertical axis represents the depth dimension and the horizontal axis shows OCT signal amplitude on a logarithmic scale with stronger signal to the right. The reflection of the air-tear interface 210 produces a peak 215 in the waveform signal. This portion of the waveform of the peak is broadened at the base posteriorly because of the addition of the reflection signal from the tear-epithelium interface reflection. The reflection from the tear-epithelium interface is not strong enough to form a separate peak because of its proximity to the stronger reflection from the air-tear interface 210. The tear film is typically less than 5 μm thick and cannot usually be resolved from an OCT image because the OCT system used in this example has a 5 μm full-width-half-maximum resolution. However, tear film+epithelial thickness can be measured between the air-tear interface peak 215 and the anterior Bowman's layer boundary peaks 225 with higher accuracy. In some embodiments, the tear film+corneal thickness can be measured between the air-tear interface peak 215 and the endothelial peak 235. Both of these thickness measurements can provide information for tear film dynamic analysis and understanding.

As an alternative to the OCT circular scan path 102, a line scan (horizontal, vertical, or any orientation) can be used to image the central tear film. The circular scan path 102 is preferred because it maintains a relatively constant OCT beam incidence angle throughout the OCT scan; thus providing more uniform and relatively constant reflectance amplitudes for the air-tear interface 210 and the different corneal layers in image 240. Constant characteristic reflectance amplitudes are advantageous because they make automated segmentation and analysis of these layers easier and more effective.

Tear Meniscus Imaging

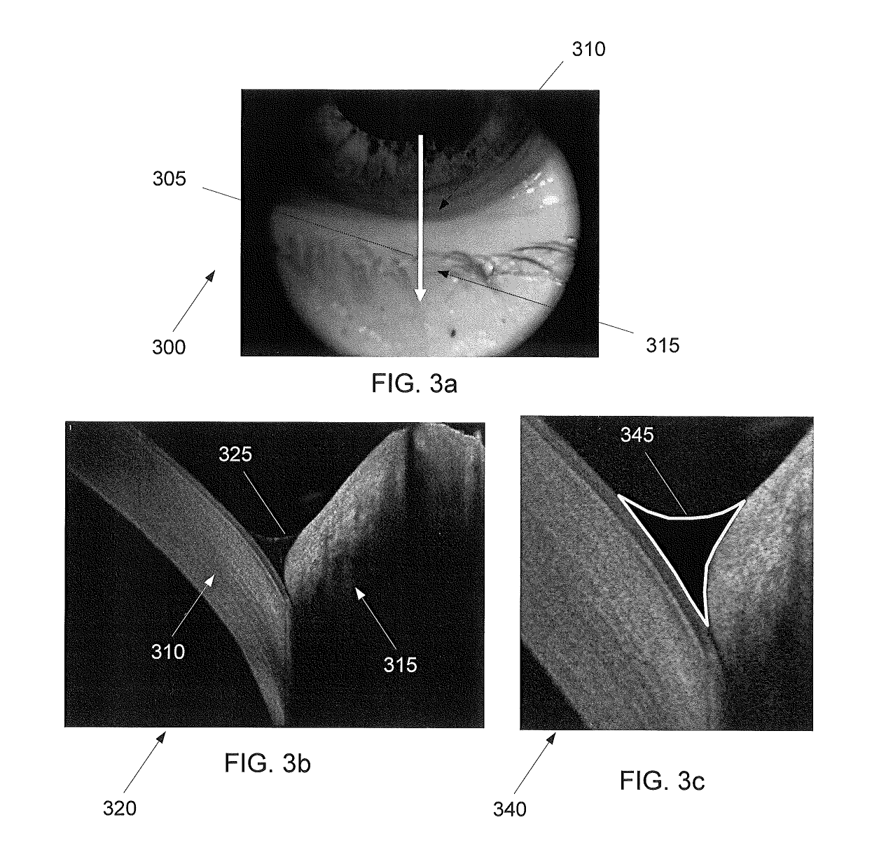

FIGS. 3a, 3b, and 3c show a representation of a vertical OCT scan and the corresponding cross-sectional OCT images of the lower tear meniscus. Image 300 shows an image that can be used to show the vertical OCT scan path 305 centered at the junction between the inferior cornea 310 (6 o'clock position) and inferior lid 315. Image 320 in FIG. 3b is an averaged cross-section image from multiple consecutive OCT scans using the vertical OCT scan path 305. The air-meniscus interface reflection 325 can be seen as the relatively bright line in image 320. The tear meniscus cross-section area 345 in FIG. 3c is the dark area defined by the air-meniscus interface 325, the inferior cornea 310 and the inferior eye lid 315. Image 340 is a magnified view of image 320 to better show the tear meniscus region 345 as outlined in white.

In some embodiments, 4 or more consecutive line scans are registered and averaged to generate the averaged cross-section image 320. Frame averaging enhances image quality by reducing speckle and unwanted background noise. This averaging method can be applied to both the circular central corneal scan path 102 to image the tear film in FIGS. 1 and 2, and the vertical line scan 305 to image the lower tear meniscus as described in FIG. 3. With a scan rate of 70,000 A-scans per second and 1024 A-scans per line, it would take less than 0.059 seconds to complete 4 consecutive OCT scans, either OCT circular scan 102 or OCT vertical scan 305. Motion error is minimal during the short acquisition period of these consecutive scans.

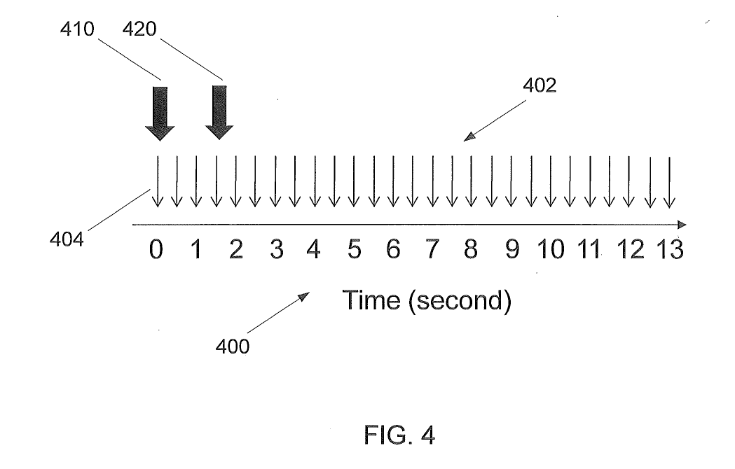

FIG. 4 shows an exemplary schematic of a timing diagram for a method for time-lapse OCT. The post-blink dynamics of the tear meniscus and tear film can be imaged using a time-lapse series. Separate OCT image series can be taken to evaluate the tear film and the tear meniscus. According to some embodiments of the present invention, the scan sequence 400 can be performed as a time-lapse series at 0.5 second intervals for a total of 13 seconds. Each thin arrow 402 in the series 400 represents 4 consecutive cross-sectional images (either tear film or tear meniscus scans). In FIG. 4, at a small fraction of a second in time after the initial scan 404, a computer or any user interface can generate a voice or other user interactive method for trigger 512 at time 410 to signal the subject to close the eye being imaged. A subsequent interactive signal from trigger 512 at time 420 can be invoked at approximately 1.5 seconds later to signal the subject to open the subject eye. Assuming there is less than 1 second of delay between the interactive means 410 or 420 and the subject reaction, a 13-second OCT scan series is capable of capturing at least 10 seconds of OCT data after the subject eye reopens. A 10-second of OCT image series provides useful information to understand and to evaluate the tear film and tear meniscus dynamics.

For tear film imaging as described with respect to FIGS. 1 and 2, tear film is formed shortly after each blink and then decays gradually by gravity until the surface tension is overcome and the smooth film breaks up. For tear meniscus imaging as described in FIG. 3, the tear meniscus is decreased by a blink and gradually increases during the inter-blink interval as the tear film drains into it. The OCT time-lapse series described in FIG. 4 can be used to provide measurements of post-blink dynamics for both the tear film and tear meniscus. The end of the blink is defined by the reopening of the eye as indicated by the reappearance of the cornea or tear meniscus after eye opening signal 420; this point in time can be defined as the time zero. For tear film evaluation, the tear+epithelial thickness can be automatically measured by computer software and averaged over the circular scan 102 at each time point 402 in the series. The change in tear+epithelial thickness between time zero and 10 seconds thereafter can be recorded as the total change. The time at which half of the change occurs can be recorded as the half time. For tear meniscus evaluation, the tear meniscus area 345 can be used as a meaningful measure. The region 345 can be automatically segmented by computer software from image 320 or image 340 to produce meaningful measures to capture the characteristics and the changes during the time sequence. In some embodiments, the detection of the tear meniscus area 345 can be performed semi-automatically. In this approach, the tear meniscus area 345 on the first OCT vertical scan 305 from image 340 can be outlined as a polygon by a clinician or a human reader, and the subsequent tear meniscus region can then be measured automatically by computer software at each time point 402. Similar to the evaluation of tear+epithelial thickness, the initial tear meniscus cross-sectional area can be measured at time zero and the final area can be measured 10 seconds thereafter. The total change between the initial and final time points can be evaluated, such as by taking the difference between these time points. The time at which half of the change occurs can be recorded as the half time.

Subjects with dry eye or dysfunctional tear syndrome can be diagnosed by these post-blink tear dynamic measures. For tear+epithelial thickness, these subjects are likely to have smaller tear film thickness total change and shorter tear film half time. For tear meniscus area evaluation, these subjects are likely to have smaller tear meniscus initial and final areas, smaller tear meniscus total change, and shorter tear meniscus half time.

Both the tear film scan described in FIGS. 1 and 2 and the tear meniscus scan described in FIG. 3 are preferably performed with the subject's head resting on a chin/forehead rest and the eye gazing on a fixation target coaxial with the optical axis of the OCT system, as is commonly performed in commercial OCT system.

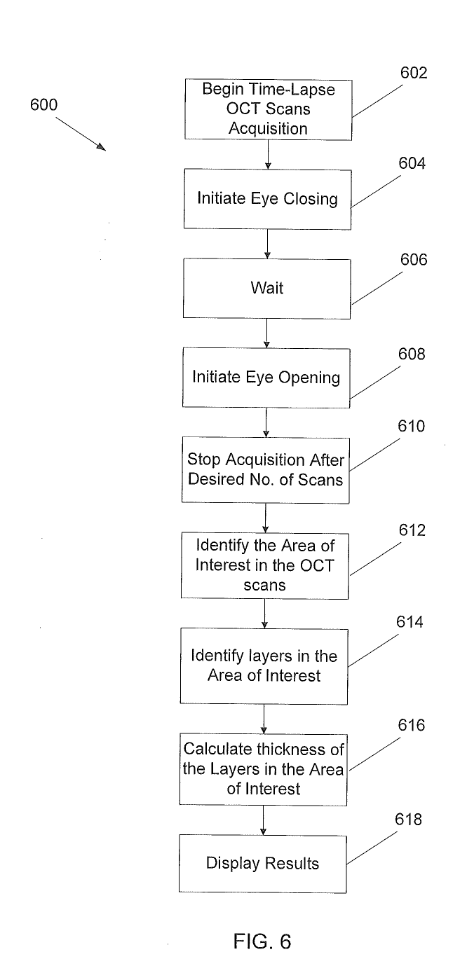

FIG. 6 illustrates an embodiment of an acquisition procedure 600 that can be performed by computer 508 according to some embodiments of the present invention. As shown in FIG. 4, step 602 begins the time-lapse OCT series acquisition procedure by continuously acquiring a plurality of OCT scans until the desired number of OCT scans are captured, the stop acquisition step 610. The OCT scans can, for example, be the circular scan 102 around a target area such as pupil 104 as illustrated in FIG. la or a vertical scan 305 as illustrated in FIG. 3a. Then, step 604 initiates the eye closing of a patient. Computer 508 performs this task by directing trigger 512 to alert the patient to close eye sample 509. In FIG. 4, this time is illustrated as time 410. As discussed above, in step 606 a wait of a fraction of one or more seconds is performed before the patient opens eye sample 509. In step 608, trigger 512 is directed by computer 508 to signal the patient to open eye sample 509 at time 420. When the desired number of OCT scans in the series is captured, the acquisition stops, step 610, and further processing will be performed on these OCT scans. In step 612, the area of interest is identified in the OCT scans series acquired beginning from step 602. In step 614, layers are identified in the area of interest for further processing. In step 616, the thicknesses of the layers located in the area of interest are then calculated. Finally, in step 618 the results are displayed on display 510.

It should be appreciated that alternative and modifications apparent to one of ordinary skills in the art can be applied within the scope of the present invention. For instance, the OCT speed, scan length, scan density, scan duration, scan interval, series length can be varied from the specific embodiments disclosed herein. Also, the tear film+corneal thickness can be used instead of the tear film+epithelium thickness in measuring the tear film dynamics and other clinically meaningful combinations of layers of interests.

We claim:

1. An imaging device, comprising:
an OCT imager;
a trigger;
a computer coupled to the OCT imager and the trigger, the computer executing instructions for:
acquiring a plurality of OCT data scans with the OCT imager at different time intervals;
generating a first signal at the trigger to initiate closing of an object at a first time;
generating a second signal at the trigger to initiate opening of an object at a second time following the first time;
identifying an area of interest in the plurality of OCT data scans;
identifying layers in the area of interest;
calculating thickness measurements of the layers from the OCT data scans; and
displaying the thickness measurements.

2. The method of claim 1, wherein the object is an eye.

3. The method of claim 1, wherein acquiring a plurality of OCT data scans includes acquiring OCT data with a circular scan configuration.

4. The method of claim 3, wherein the circular scan can be centered at or about the center of the object.

5. The method of claim 3, wherein the circular scan configuration can be repeated at least one time and the OCT data scans are data averaged.

6. The method of claim 1, wherein acquiring a plurality of OCT data scans includes acquiring OCT data with a vertical scan configuration.

7. The method of claim 6, wherein the object is an eye and wherein the vertical scan configuration can be centered at or about a junction between an inferior cornea and an inferior lid of the eye.

8. The method of claim 6, wherein the vertical scan configuration can be repeated at least one time and the OCT data scans are data averaged.

9. The method of claim 1, wherein the different time intervals are substantially equally spaced in time.

10. The method of claim 9, wherein the different time intervals can be 0.5 seconds.

11. The method of claim 1, wherein the plurality of OCT data scans provides data for at least 10 seconds.

12. The method of claim 1, wherein the object is an eye and wherein the area of interest can be the thickness between a tear layer and an epithelial layer.

13. The method of claim 1, wherein the object in an eye and wherein the area of interest can be a cross-section area defined by boundaries between an air-meniscus interface, an inferior cornea and an inferior eye lid.

14. The method of claim 1, wherein the thickness measurements can be differences between the area of interest at the time interval immediately after the eye opening motion and the last time interval.

15. The method of claim 1, wherein the thickness measurements can be differences between the area of interest at the time interval immediately after the eye opening motion and an half-time interval; the half-time interval is the mid-point between the time interval immediately after the eye opening motion and the last time interval.

16. A method comprising:
acquiring a plurality of OCT data scans at different time intervals;
generating a first signal to initiate closing of an object at a first time;
generating a second signal to initiate opening of an object at a second time following the first time;
identifying an area of interest in the plurality of OCT data scans;
identifying layers in the area of interest;
calculating thickness measurements of the layers from the OCT data scans; and
displaying the thickness measurements.

17. The method of claim 16, wherein the object is an eye.

18. The method of claim 16, wherein acquiring a plurality of OCT data scans includes acquiring OCT data with a circular scan configuration.

19. The method of claim 18, wherein the circular scan can be centered at or about the center of the object.

20. The method of claim 18, wherein the circular scan configuration can be repeated at least one time and the OCT data scans are data averaged.

21. The method of claim 16, wherein acquiring a plurality of OCT data scans includes acquiring OCT data with a vertical scan configuration.

22. The method of claim 21, wherein the object is an eye and wherein the vertical scan configuration can be centered at or about a junction between an inferior cornea and an inferior lid of the eye.

23. The method of claim 21, wherein the vertical scan configuration can be repeated at least one time and the OCT data scans are data averaged.

24. The method of claim 16, wherein the different time intervals are substantially equally spaced in time.

25. The method of claim 24, wherein the different time intervals can be 0.5 seconds.

26. The method of claim 16, wherein the plurality of OCT data scans provides data for at least 10 seconds.

27. The method of claim 16, wherein the object is an eye and wherein the area of interest can be the thickness between a tear layer and an epithelial layer.

28. The method of claim 16, wherein the object in an eye and wherein the area of interest can be a cross-section area defined by boundaries between an air-meniscus interface, an inferior cornea and an inferior eye lid.

29. The method of claim 16, wherein the thickness measurements can be differences between the area of interest at the time interval immediately after the eye opening motion and the last time interval.

30. The method of claim 16, wherein the thickness measurements can be differences between the area of interest at the time interval immediately after the eye opening motion and an half-time interval; the half-time interval is the mid-point between the time interval immediately after the eye opening motion and the last time interval.
